(19) United States
(12) Patent Application Publication
(10) Pub. No.: US 2026/0241141 A1
(43) Pub. Date: Aug. 20, 2026

(54) INTERVENTIONAL SURGICAL INSTRUMENT OPERATING SYSTEM FOR GENERATING COMPOSITE MOTION OF INTERVENTIONAL SURGICAL INSTRUMENTS

(71) Applicant: TSINGHUA UNIVERSITY, Beijing (CN)

(72) Inventors: Zhelong WANG, Beijing (CN); Gangtie ZHENG, Beijing (CN)

(21) Appl. No.: 19/533,329

(22) Filed: Feb. 9, 2026

(30) Foreign Application Priority Data
Feb. 11, 2025 (CN) 202510151427.X

(51) Int. Cl. A61M 25/01; A61B 17/00; A61M 25/09

(52) U.S. Cl. CPC A61M 25/0113; A61M 25/09041; A61B 2017/00292; A61M 2205/332; A61M 2205/3327

(57) ABSTRACT

The present disclosure discloses an interventional surgical instrument operating system for vascular interventional surgeries, which can be used in interventional surgical treatments for subcategories such as cardiovascular, cerebrovascular, peripheral vascular, and hepatobiliary diseases. The characteristic of the system is that: the execution end, which manipulates the interventional surgical instruments, drives the interventional surgical instruments to achieve composite motion through the rotation and vertical motion of friction wheels with convex-concave helical lines distributed on their outer surfaces. The doctor operates the master end, where a circular operating lever supported at both ends generates composite motion commands combining linear and rotational motion of the interventional surgical instrument. The communication and control system performs signal transmission and control between said master end and execution end.

### CROSS-REFERENCE TO RELATED APPLICATION

This application claims priority to Chinese Patent Application No. 202510151427.X filed on Feb. 11, 2025, the entire disclosure of which is incorporated herein by reference.

### FIELD

The present disclosure belongs to the field of surgical robots, and particularly relates to an interventional surgical instrument operating system for generating composite motion of interventional surgical instruments.

### BACKGROUND

Interventional surgical instrument operating systems include a master end (remote control end) and an execution end (slave end). A surgeon controls the execution end via the master end to manipulate the motion of interventional surgical instruments inside the human body. Interventional surgical instruments include guidewires, catheters, balloons, thrombectomy devices, etc. To reach lesions in complex vascular networks, interventional surgical instruments need to perform linear motion while simultaneously performing rotational motion, i.e., forming a composite motion combining linear and rotational movements. The operation to achieve this motion is also called a push-twist operation.

Interventional surgical instrument operating systems, also known as interventional surgical assistance devices (commonly called surgical robots), still have many problems and shortcomings. Currently, most master ends, both domestic and international, use two joysticks to separately control the linear motion and rotational motion of the interventional surgical instrument. Doctors need to use both hands to control these two joysticks separately, which prevents synchronous operation. This greatly differs from the way doctors use one hand to perform linear pushing plus rotating operations on catheters and guidewires during vascular interventional surgery, thus violating the surgical experience and operational intuition accumulated by doctors during long-term practice. This forces doctors to undergo specialized training to operate correctly. For experienced senior doctors, the valuable experience accumulated over time, on the contrary becomes an obstacle to mastering new operation methods, easily leading to mis-operations and causing a decline in surgical quality. Corresponding to the master end's operation method, the execution ends of most current interventional surgical operating systems operate linear motion (delivery) and rotational motion (twisting) separately, i.e., when performing linear motion, rotational motion operation cannot be performed. In summary, a major problem with current interventional surgical operating systems is that the design of the master end and execution end separates delivery and twisting operations apart. Patents such as U.S. Pat. No. 10,245,112 from Corindus, Inc., and Chinese patents CN105534599A and CN113729960A are typical examples of such designs. Apart from using different joysticks at the master end to operate the rotational and linear motions of the interventional surgical instrument separately, these patents also decouple the instrument's linear and rotational motions at the execution end, using different drive devices to achieve the motions, thus unable to achieve ideal push-twist motion, which severely limits the ability to navigate through a complex vasculature.

### SUMMARY

The present disclosure aims to solve at least one of the technical problems existing in the prior art. To this end, the present disclosure proposes an interventional surgical instrument operating system capable of generating composite motion for interventional surgical instruments.

The interventional surgical instrument operating system includes an execution end. This execution end can achieve composite motion of simultaneous linear and rotational motion of the interventional surgical instrument, i.e., achieving ideal push-twist motion, with advantages such as good driving effect, high accuracy, and strong reliability. A matching master end, also called a remote control end, provides a simulation of the guidewire torque device used by doctors, capable of directly generating push-twist motion commands transmitted to the execution end via a communication module.

To achieve the above objective, according to an embodiment of a first aspect of the present disclosure, an execution end is proposed, said execution end including: a plurality of first friction wheels arranged along a first horizontal direction; a plurality of second friction wheels arranged along the second horizontal direction, the plurality of said first friction wheels being correspondingly and oppositely disposed relative to the plurality of said second friction wheels and adapted to jointly grip an interventional surgical instrument, outer surfaces of the plurality of said first friction wheels and the plurality of said second friction wheels being distributed with grooves forming helices with a certain helix angle, the wheel helix angle usually being less than ninety degrees, i.e., acute, these grooves being step-shaped, constituting a layer of uneven helical grooves on the friction wheel surface, serving to prevent slippage between the interventional surgical instrument and the friction wheels while simultaneously enabling synchronous implementation of rotational motion and forward/backward linear motion, the gap between each pair of friction wheels being adjustable to adapt to the operation of interventional surgical instruments with different diameters. For convenience of description, this friction wheel that simultaneously generates composite linear and rotational motion and has anti-slip function is simply referred to as a friction wheel, and the grooves forming helices with a certain helix angle are simply referred to as helices. On the friction wheels, the helix angles of the plurality of said helices are all the same, the helical directions of the helices on adjacent two said first friction wheels are opposite, the helical directions of the helices on adjacent two said second friction wheels are opposite, and the helical directions of the helices on opposing said first friction wheel and said second friction wheel are opposite.

The friction wheels are mounted on rotating spline shafts; correspondingly, the interior of the friction wheels is gear-shaped, so that there is no relative rotational motion between the friction wheels and the shaft, i.e., the rotational motion of the friction wheels is the same as that of the shaft, but the friction wheels can move up and down along the rotating spline shaft while performing rotational motion, thereby achieving simultaneous rotational and up-down linear motion under the drive of a drive device.

The execution end according to the embodiment of the present disclosure can achieve composite motion of simultaneous forward/backward motion and left/right rotation of the interventional surgical instrument, i.e., the ideal push-twist motion, and has the function of preventing slippage of the interventional instrument, with advantages such as simulating manual operation by doctors, high accuracy, and strong reliability.

Furthermore, the execution end according to the above embodiment of the present disclosure may also have the following additional technical feature.

According to an embodiment of the present disclosure, said execution end further includes: a first lifting base, said plurality of first friction wheels being provided on said first lifting base, said first lifting base being liftable; a second lifting base, said plurality of second friction wheels being provided on said second lifting base, said second lifting base being liftable; a rotation drive device, said rotation drive device being respectively connected in drive to said first lifting base and said second lifting base and adapted to drive said first lifting base and said second lifting base to lift and lower in opposite directions.

Said surgical execution end further includes: a gap adjustment drive device, said gap adjustment drive device being provided on said base and connected in drive to said movable base.

According to an embodiment of the present disclosure, said rotation drive device includes: an up-down motion drive motor; a crank, the center of said crank being connected to the motor shaft of said up-down motion drive motor; a first connecting lever, both ends of said first connecting lever being pivotally connected to one end of said crank and said first lifting base respectively; a second connecting lever, both ends of said second connecting lever being pivotally connected to the other end of said crank and said second lifting base respectively.

According to an embodiment of the present disclosure, said execution end further includes a radial limiting element, said interventional surgical instrument being adapted to fit within said radial limiting element so as to be axially movable and circumferentially rotatable, said radial limiting element being adapted to limit radial movement of said interventional surgical instrument.

According to an embodiment of the present disclosure, said execution end further includes: a first rotation drive device, said first rotation drive device being respectively connected in drive to said plurality of first friction wheels, said first rotation drive device being adapted to drive said plurality of first friction wheels to rotate at the same speed and in the same direction; a second rotation drive device, said second rotation drive device being respectively connected in drive to said plurality of second friction wheels, said second rotation drive device being adapted to drive said plurality of second friction wheels to rotate at the same speed and in the same direction, and the rotation directions of said plurality of first friction wheels being opposite to those of said plurality of second friction wheels.

Considering that interventional surgeries often involve delivering multiple interventional surgical instruments to the patient's lesion site and operating on the lesion, according to an embodiment of a second aspect of the present disclosure, an execution end is proposed, said execution end including: a second interventional surgical instrument (usually a catheter) operation module, said second interventional surgical instrument operation module including the execution end according to the embodiment of the first aspect of the present disclosure; and/or a first interventional surgical instrument (usually a guidewire) operation module, said first interventional surgical instrument operation module including the execution end according to the embodiment of the first aspect of the present disclosure.

A third interventional surgical instrument operation module is also integrated on the second interventional surgical instrument operation module. This module consists of two sets of symmetrically placed rollers with opposite rotation directions, used for delivering interventional surgical instruments that do not require rotational motion, such as balloons for delivering vascular stents.

The interventional surgical instrument operating system according to the embodiment of the present disclosure, by utilizing the execution end according to the embodiment of the second aspect of the present disclosure, can achieve simultaneous forward/backward motion and rotational motion of the interventional surgical instrument, i.e., ideal push-twist motion, can prevent slippage of the interventional instrument, and has advantages such as simulating manual operation by doctors, high accuracy, and strong reliability.

According to an embodiment of the present disclosure, the master end in said interventional surgical instrument operating system further includes: a circular operating lever, a linear motion module with a displacement sensor, a rotational motion module with a rotary encoder, a DC motor or a rotational electromagnetic damper providing rotational direction force feedback, a linear motor providing linear direction force feedback, a low-resistance transmission mechanism decoupling forward/backward linear motion and rotational motion, and a frame. The displacement sensor detects the hand's movement direction and amplitude, with forward and backward movements corresponding to the forward and backward movement directions of the interventional surgical instrument driven by the execution end. The distance the front end moves forward defines the speed at which the execution end drives the interventional surgical instrument forward. The encoder's output angle and direction are consistent with the rotation angle and direction of the interventional surgical instrument driven by the execution end.

According to an embodiment of the present disclosure, the linear motor is a voice coil motor.

According to an embodiment of the present disclosure, the circular operating lever included in the master end of said interventional surgical instrument operating system is characterized by: a circular operating lever simulating an interventional surgical instrument torque device, its surface knurled or having groove patterns to prevent slippage of the hand in contact. A wrist support part is also installed below the operating lever, said wrist support part being provided on said frame.

According to an embodiment of the present disclosure, the master end of said interventional surgical instrument operating system further includes a control box, on which is installed a scaling factor device for proportionally scaling the motion speed ratio, angular velocity ratio, displacement amplitude ratio, and rotation angle ratio of the interventional surgical instrument motion commands generated by the master end for the execution end, allowing the user to choose to increase or decrease the motion speed and amplitude of the interventional surgical instrument according to specific circumstances with such an effect as increasing the operation resolution.

According to an embodiment of the present disclosure, the control box internally contains a controller, said controller communicating via said communication device with said linear motor, said displacement detection device, said drive motor, said angle detection device, and said execution end, said controller processes the linear delivery resistance power obtained by the execution end to control said linear motor to provide axial feedback resistance for the axial movement of said operating lever, said controller processes the rotational resistance power obtained by the execution end to control said drive motor or said rotational electromagnetic damper to provide rotational feedback resistive torque for the rotation of said operating lever, and said controller is adapted to control said execution end based on said axial displacement direction, said axial displacement distance, and said rotation angle.

According to an embodiment of the present disclosure, the communication device for establishing signal communication connection between the master end and the execution end can be wired or wireless.

Additional aspects and advantages of the present disclosure will be provided in the following description, and will become apparent from the following description, or be understood through practice of the present disclosure.

### BRIEF DESCRIPTION OF THE DRAWINGS

The above and/or additional aspects and advantages of the present disclosure will become apparent and readily understood from the description of embodiments in conjunction with the following drawings, wherein:

FIG. 1 is a schematic diagram of an interventional surgical instrument operating system according to an embodiment of the present disclosure.

FIG. 2 is a schematic diagram of a catheter operation box and a catheter operation module of an execution end according to an embodiment of the present disclosure.

FIG. 3 is a schematic diagram of an interventional surgical execution end of a catheter operation module of an execution end according to an embodiment of the present disclosure.

FIG. 4 is a schematic diagram of an execution end according to an embodiment of the present disclosure.

FIG. 5 is a schematic diagram of an execution end according to an embodiment of the present disclosure.

FIG. 6 is a schematic diagram of an execution end according to an embodiment of the present disclosure.

FIG. 7 is a schematic diagram of an execution end according to an embodiment of the present disclosure.

FIG. 8 is a schematic diagram of a guidewire/balloon operation box, a guidewire operation module, and a balloon catheter operation module of an execution end according to an embodiment of the present disclosure.

FIG. 9 is a schematic diagram of a guidewire operation module and a balloon catheter operation module of an execution end according to an embodiment of the present disclosure.

FIG. 10 is a schematic diagram of an execution end of a guidewire operation module of an execution end according to an embodiment of the present disclosure.

FIG. 11 is a schematic diagram of an execution end of a balloon catheter operation module of an execution end according to an embodiment of the present disclosure.

FIG. 12 is a schematic diagram of an execution end of a balloon catheter operation module of an execution end according to an embodiment of the present disclosure.

FIG. 13 is a schematic diagram of an execution end according to an embodiment of the present disclosure.

FIG. 14 is a schematic diagram of a Y-valve positioning device of a balloon catheter operation module of an execution end according to an embodiment of the present disclosure.

FIG. 15 is a schematic diagram of a roller cart and a passive damping mechanical arm of an execution end according to an embodiment of the present disclosure.

FIG. 16 is a schematic diagram of a passive damping mechanical arm of an execution end according to an embodiment of the present disclosure.

FIG. 17 is a schematic diagram of a master end of an interventional surgical instrument operating system according to an embodiment of the present disclosure.

FIG. 18 is a motion schematic diagram of an interventional surgical instrument achieved by an execution end of an interventional surgical instrument operating system according to an embodiment of the present disclosure.

FIG. 19 is an interventional surgical instrument placement guiding device included in an execution end according to an embodiment of the present disclosure, for preventing radial up-down motion of the interventional surgical instrument.

Reference numerals: interventional surgical instrument operating system 1, catheter operation box 100, catheter operation module 110, guidewire/balloon operation box 200, guidewire operation module 210, balloon catheter operation module 220, Y-valve positioning device 230, elastic claw 231, passive damping mechanical arm 300, arm segment 310, adapter base 311, first bent tube 312, second bent tube 313, third bent tube 314, connection base 320, support tube 330, connection tube 340, roller cart 400, support base 410, support lever 420, roller 430, interventional surgical execution end 1000, first friction wheel 1011, second friction wheel 1012, helix 1013, flat key shaft 1014, driven gear 1015, friction wheel base 1016, first lifting base 1021, second lifting base 1022, up-down motion drive motor 1031, crank 1032, first connecting lever 1033, second connecting lever 1034, radial limiting element 1040, base 1050, fixed base 1061, movable base 1062, gap adjustment drive device 1070, first rotation drive device 1081, second rotation drive device 1082, master end 20, operating lever 21, linear motor 22, displacement detection device 23, drive motor 24, ball spline structure 25, controller 26, hand support pad 27, communication device 30, Y-valve 2.

### DETAILED DESCRIPTION

The present application is made based on the inventor's discovery and understanding of the following facts and problems:

Interventional surgical instrument operating systems include a master end and an execution end. Surgeons control the operation of the execution end via the master end. The drive mechanism of the execution end drives interventional surgical instruments such as guidewires and catheters.

In the execution end of related art interventional surgical instrument operating systems, friction wheels are used to drive the interventional surgical instrument. The friction wheels have two motion forms: rotational motion and up-down motion. To simultaneously achieve linear and rotational motion of the interventional surgical instrument, grooves forming helices with a certain helix angle need to be arranged on the outer surface of the friction wheels. These grooves are step-shaped, constituting a layer of uneven helical grooves on the friction wheel surface, and to avoid slippage between the interventional surgical instrument and the friction wheels, which affects the driving effect and accuracy for the interventional surgical instrument.

Furthermore, in some related art interventional surgical instrument operating systems, to simulate the doctor's hand operation habits and make the master end more consistent with the doctor's hand operation habits, the execution end can also feed back the resistance encountered by the interventional surgical instrument to the master end, simulating the resistance encountered by the interventional surgical instrument when the doctor operates the master end. Or in other words, the motion signals generated by the master end need to be one-to-one corresponding motion signals with the execution end motion, and be able to reproduce the linear motion resistance and rotational motion resistance (resistive torque) experienced by the interventional surgical instrument operated by the execution end.

The inventor believes that: the core of achieving composite motion of simultaneous linear and rotational motion of the interventional surgical instrument, i.e., the ideal push-twist motion, is that the friction wheels gripping the interventional surgical instrument can simultaneously achieve radial motion (up-down linear motion) perpendicular to the linear motion direction of the interventional surgical instrument and rotational motion. The opposite up-down motion of a pair of opposing friction wheels can achieve rotational motion of the interventional surgical instrument, while the opposite rotational motion of this pair of friction wheels can achieve linear motion of the interventional surgical instrument. A structure that can realize this motion mode of the friction wheels is to install the friction wheels on spline shafts, thus allowing simultaneous linear and rotational motion of the friction wheels. Correspondingly, the operating lever of the master end uses a spline shaft to connect to a rotary encoder and a linear motion position sensor at the two ends of the operating lever respectively, thereby being able to simultaneously generate linear and rotational motion control command signals. The aforementioned linear motor and DC motor/rotational electromagnetic damper only provide the capability to simulate the resistance and resistive torque experienced by the interventional surgical instrument inside the human body, in order to provide the doctor operating the master end with the feeling of directly manually operating the interventional surgical instrument, i.e., implementing force feedback.

When driving the interventional surgical instrument's rotation and linear motion encounters resistance/resistive torque, the input power of the motors driving the friction wheels increases. To obtain the resistance and resistive torque (rotational resistance) experienced by the interventional surgical instrument, force sensors need to be used for calibration, establishing the relationship between resistance/resistive torque and input power change, forming a transformation function. One implementation method is to use the execution end to push and rotate a rigid lever with a six-degree-of-freedom force sensor installed in the middle, with the front end of the lever immersed in a container filled with magnetorheological fluid. By controlling the damping value of the magnetorheological fluid, different resistances and resistive torques are obtained, establishing the relationship function with the output value of the force sensor, thereby obtaining the relationship function between motor input power and resistance/resistive torque. Similarly, the same method is used at the master end to obtain the relationship function between the linear motor/DC motor/rotational electromagnetic damper input power and the resistance/resistive torque it generates.

In actual operation, since the motors driving the delivery and rotation mechanisms also require electrical power, it is necessary to first operate the interventional surgical instrument to perform linear and rotational motion before it enters the patient's body, obtain the motor input power values, and use them as initial values. When calculating the resistance and resistive torque encountered by the interventional surgical instrument after entering the patient's body, subtract them from the measured power values.

For such interventional surgical instrument operating systems, slippage between the friction wheels and the interventional surgical instrument can seriously affect the accuracy of resistance feedback, causing the doctor to be unable to accurately judge the resistance experienced by the interventional surgical instrument during surgical operation, severely affecting the progress of the surgical procedure.

The following describes embodiments of the present disclosure in detail, with examples of the embodiments shown in the accompanying drawings. Throughout the drawings, the same or similar reference numerals refer to the same or similar elements or elements having the same or similar functions. The embodiments described below with reference to the drawings are exemplary and are intended to explain the present disclosure only, and should not be construed as limiting the present disclosure.

In the description of the present disclosure, it should be understood that terms such as “center”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “counterclockwise”, “axial”, “radial”, “circumferential”, etc., indicate orientations or positional relationships based on those shown in the accompanying drawings. They are used only for the convenience of describing the present disclosure and simplifying the description, and do not indicate or imply that the referred device or element must have a specific orientation, be constructed and operated in a specific orientation, and therefore should not be construed as limiting the present disclosure. Furthermore, the terms “first” and “second” are used for descriptive purposes only and are not to be construed as indicating or implying relative importance or implicitly indicating the number of technical features referred to. Thus, features defined with “first” and “second” may explicitly or implicitly include one or more of those features. In the description of the present disclosure, “a plurality of” means two or more, unless otherwise specified.

In the description of the present disclosure, it should be noted that, unless otherwise explicitly specified and defined, the terms “mounted”, “connected”, and “coupled” are to be interpreted broadly. For example, they may be fixed connections, detachable connections, or integral connections; they may be mechanical connections or electrical connections; they may be direct connections or indirect connections via an intermediate medium, or they may be internal connections between two elements. The specific meanings of the above terms in the present disclosure can be understood by those skilled in the art in light of specific circumstances.

In the description of the present disclosure, guidewires and catheters, two commonly used interventional surgical instruments, are taken as examples for illustration, but in actual surgical operations, other types of interventional surgical instruments may be applicable.

The interventional surgical execution end 1000 according to an embodiment of the present disclosure is described below with reference to the drawings.

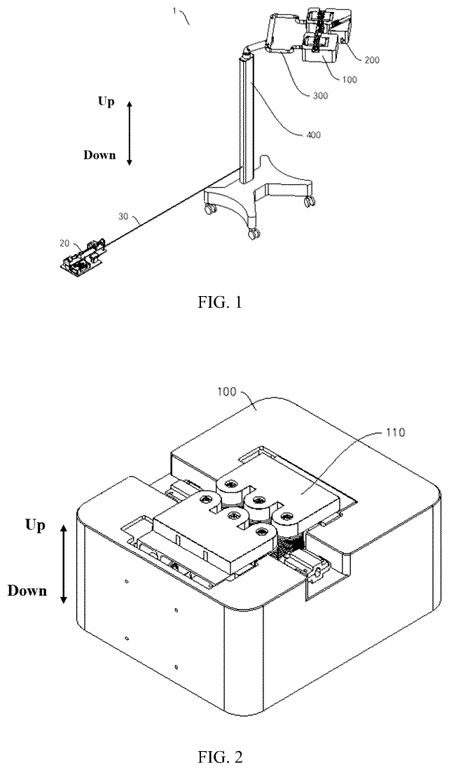

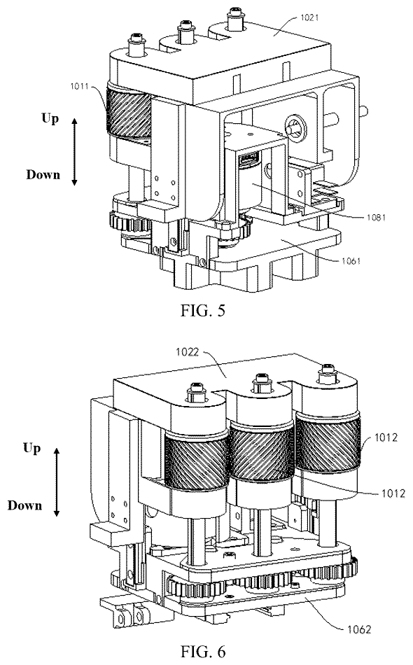

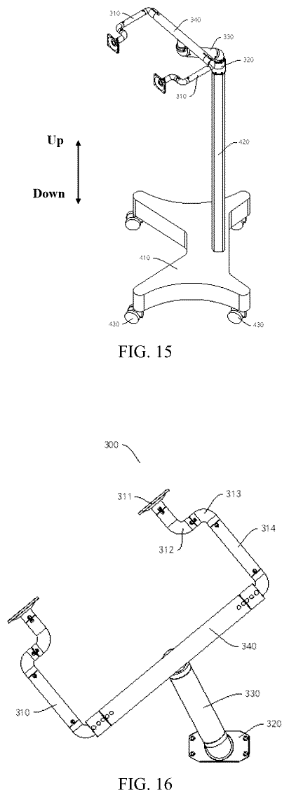

First, the interventional surgical instrument operating system 1 according to an embodiment of the present disclosure is described with reference to FIGS. 1-17.

The interventional surgical instrument operating system 1 according to an embodiment of the present disclosure includes a master end 20, a communication device 30, and an execution end.

The execution end communicates with the master end 20 via the communication device 30.

A surgeon operates the master end 20 to control the operation of the execution end, and the execution end drives the interventional surgical instrument.

The execution end according to an embodiment of the present disclosure includes at least a catheter operation module 110 and/or a guidewire operation module 210.

The catheter operation module 110 includes the interventional surgical execution end 1000 according to an embodiment of the present disclosure.

The guidewire operation module 210 includes the interventional surgical execution end 1000 according to an embodiment of the present disclosure.

In an exemplary embodiment of the present disclosure, the execution end 10 according to an embodiment of the present disclosure may include a catheter operation module 110, a guidewire operation module 210, and a balloon catheter operation module 220. The catheter operation module 110 is adapted to operate a catheter, the guidewire operation module 210 is adapted to operate a guidewire, and the balloon catheter operation module 220 is adapted to operate a balloon catheter. The catheter operation module 110, guidewire operation module 210, and balloon catheter operation module 220 may include the interventional surgical execution end 1000 according to the same or different embodiments of the present disclosure.

The execution end of the interventional surgical instrument operating system includes: a plurality of friction wheels for gripping and conveying an interventional surgical instrument, the outer surface of the friction wheels being provided with grooves forming a plurality of helices with an acute helix angle, the grooves being step-shaped, constituting a layer of uneven helical grooves on the friction wheel surface.

The plurality of said friction wheels include: a plurality of first friction wheels arranged along a first horizontal direction; a plurality of second friction wheels arranged along the second horizontal direction, which is parallel to the first direction, the plurality of said first friction wheels being correspondingly and oppositely disposed relative to the plurality of said second friction wheels in a second horizontal direction perpendicular to the first horizontal direction, jointly gripping the interventional surgical instrument, wherein the helical directions of the helices on the outer surfaces of all first friction wheels are opposite to those on the outer surfaces of all second friction wheels.

The execution end of the interventional surgical instrument operating system further includes a rotating spline shaft, the friction wheels being mounted on said rotating spline shaft; correspondingly, the interior of the friction wheels is gear-shaped, allowing the friction wheels to move up and down along the rotating spline shaft while performing rotational motion.

The execution end of the interventional surgical instrument operating system further includes: a fixed base; a movable base; lifting bases; and a bottom plate of an actuator housing, the fixed base being fixed on the inner surface of the bottom plate of the actuator housing, the movable base being mounted on a linear motion slide rail, said linear motion slide rail being fixed below on said inner surface of the bottom plate of the actuator housing, said lifting bases including: a first lifting base, said plurality of first friction wheels being provided on said first lifting base, said first lifting base being liftable, driving said plurality of first friction wheels to move up and down respectively along said plurality of spline-type rotating shafts, the first lifting base being mounted on the fixed base, the plurality of spline-type rotating shafts being mounted on said fixed base; a second lifting base, said plurality of second friction wheels being provided on said second lifting base, said second lifting base being liftable, driving said plurality of second friction wheels to move up and down respectively along said plurality of spline-type rotating shafts, the second lifting base being mounted on said movable base, the plurality of spline-type rotating shafts being mounted on said movable base, wherein the up-down movement directions of said plurality of first friction wheels are opposite to those of said plurality of second friction wheels, forming an interleaved up-down motion, which, combined with the synchronous rotational motion of the friction wheels, forms the composite rotational and linear motion of the interventional surgical instrument.

The execution end of the interventional surgical instrument operating system includes a friction wheel rotation drive device, the friction wheel rotation drive device including: a first rotation drive device, said first rotation drive device including a motor and a gear transmission device, respectively connected in drive to said plurality of first friction wheels, said first rotation drive device driving said plurality of first friction wheels to rotate at the same speed and in the same direction; a second rotation drive device, said second rotation drive device including a motor and a gear transmission device, respectively connected in drive to said plurality of second friction wheels, said second rotation drive device driving said plurality of second friction wheels to rotate at the same speed and in the same direction, and the rotation directions of said plurality of first friction wheels being opposite to those of said plurality of second friction wheels.

The execution end of the interventional surgical instrument operating system includes a friction wheel up-down motion drive device, which converts the rotational motion of a drive motor into linear up-down motion driving the lifting bases via a pair of crank-link mechanisms, the friction wheel up-down motion drive device including: a crank, the center of said crank being connected to the motor shaft of said up-down motion drive motor; a first connecting lever, both ends of said first connecting lever being pivotally connected to one end of said crank and said first lifting base respectively, driving said first lifting base to move up and down; a second connecting lever, both ends of said second connecting lever being pivotally connected to the other end of said crank and said second lifting base respectively, driving said second lifting base to move up and down.

The execution end of the interventional surgical instrument operating system includes a gap adjustment drive device, said gap adjustment drive device including: a linear motion actuator, arranged parallel to said linear motion slide rail, its fixed part being fixed on the inner surface of said bottom plate of the actuator housing, its extendable part being connected to the bottom of the movable base; a displacement sensor configured to record a distance between said movable base and said fixed base.

The execution end of the interventional surgical instrument operating system further includes: a limit switch system configured to restrict the up-down motion of the friction wheels, wherein when the friction wheels move up or down to reach the maximum stroke of the lifting base on which they are mounted, a limit switch can be triggered to achieve automatic reset by an automatic reset execution end.

Said gap adjustment drive device has an automatic reset function, wherein when a limit switch is triggered, said gap adjustment drive device drives the movable base and the fixed base to be separated from each other, the displacement sensor records a separation distance, said friction wheel up-down motion drive device moves said first lifting base and said second lifting base to an initial motion position, and then the gap adjustment drive device moves the movable base back to a position before separation in the reverse direction according to the movement distance recorded by the displacement sensor, to grip the interventional surgical instrument again.

The execution end of the interventional surgical instrument operating system further includes: an interventional surgical instrument placement guiding device, said guiding device being divided into a plurality of modules, each module including a base and an upper cover, the bases of all modules being structurally connected to form an integral whole, the middle of the base and the upper cover having a groove, two opposite grooves forming a hole, the interventional surgical instrument passing through from the middle, the width of said groove being larger than the diameter of the interventional surgical instrument placed thereon, the depth of said groove being larger than the radius of the interventional surgical instrument placed thereon, said plurality of modules including a first module, a second module and a third module, the first module being installed between pairs of friction wheels arranged side by side along the first horizontal direction, the base and the upper cover forming a three-dimensional structure, the upper cover connecting a plurality of upright structures located along the second horizontal direction between adjacent friction wheels arranged along the first horizontal direction; the second and third module structures being respectively installed outside the sides of the plurality of friction wheels arranged side by side along the first horizontal direction and the second horizontal direction, being planar structures, with the upper covers located outside the side-by-side arranged friction wheels.

A method for measuring resistance and resistive torque experienced by an interventional surgical instrument includes: detecting resistance and rotational resistive torque experienced by the interventional surgical instrument delivered by the execution end; and measuring resistance including steps of: placing the interventional surgical instrument between the first and second friction wheel groups, after clamping, first measuring the input power of the up-down motion drive motor and the rotation drive motor before the interventional surgical instrument enters the patient's body as an initial input power value; measuring the input power of the up-down motion drive motor and the rotation drive motor after the interventional surgical instrument enters the patient's body; subtracting the initial input power value from the measured input power, and taking the obtained difference as a power consumed by the resistance experienced by the interventional surgical instrument; and through a conversion formula, obtaining the resistance and the resistive torque experienced by the interventional surgical instrument after entering the patient's body. The conversion formula is obtained through experiments.

An execution end includes: a first interventional surgical instrument operation module, said first interventional surgical instrument operation module including the execution end for an interventional surgical instrument operating system according to the above-described embodiments of the present disclosure; and/or a second interventional surgical instrument operation module, said second interventional surgical instrument operation module including the execution end for an interventional surgical instrument operating system according to the above-described embodiments of the present disclosure; a third interventional surgical instrument operation module, installed on the same base plate as the second interventional surgical instrument operation module, the third interventional surgical instrument operation module being configured to push interventional surgical instruments that do not require rotational twisting, the third interventional surgical instrument operation module including a plurality of third friction wheels arranged along a third horizontal direction and a plurality of fourth friction wheels arranged along the fourth horizontal direction, rotation directions of the plurality of third friction wheels being opposite to those of the plurality of fourth friction wheels. The plurality of third friction wheels and the plurality of fourth friction wheels are driven by the same drive motor via a gear set.

The execution end further includes: a roller cart, said roller cart including a support base, a support lever, and rollers, said support lever being provided on said support base, said rollers being provided on the lower surface of said support base; a joint-angle adjustable lockable mechanical arm, said mechanical arm being provided on said support lever, said mechanical arm including at least two arm segments, said first interventional surgical instrument operation module and said second interventional surgical instrument operation module being respectively provided on the two said arm segments.

An interventional surgical instrument operating system includes: a master end; a communication device, capable of establishing signal communication connection between the execution end and the master end via cable/wireless network; an execution end, said execution end being the execution end according to any one of claims 1-13, said execution end communicating with said master end via said communication device.

The master end includes: a circular operating lever; a linear motion module with a displacement sensor; a rotational motion module with a rotary encoder.

Said circular operating lever simulates a torque device for gripping an interventional surgical instrument, its surface being knurled or having groove patterns, a first end being connected to the moving part of a linear motor, a second end being connected to the rotating part of a DC motor or a rotational electromagnetic damper via a spline shaft, said operating lever being capable of moving back and forth along the spline shaft; a distance by which said operating lever moves towards the linear motor defines a speed at which the execution end manipulates the interventional surgical instrument to move forward, a greater distance corresponding to a higher speed.

The linear motion module with a displacement sensor includes: a displacement sensor; a linear motor; said displacement sensor detects the movement direction and amplitude of said operating lever, forward and backward movements corresponding to the forward and backward movement directions of the interventional surgical instrument manipulated by the execution end; said linear motor provides force feedback in the linear direction, its moving part being connected to one end of said operating lever.

The rotational motion module with a rotary encoder includes: a rotary encoder; a DC motor or a rotational electromagnetic damper; said rotary encoder measures the rotation angle and rotational angular velocity of said operating lever, the output angle direction being consistent with the rotation direction of the interventional surgical instrument manipulated by the execution end; said DC motor or rotational electromagnetic damper provides torque feedback in the rotational direction, its rotating part being connected to said operating lever via a spline.

The master end further includes: a master end control box, on which is installed a scaling factor device for proportionally amplifying or reducing a linear motion amplitude, a linear motion speed, a rotation angle, and a rotational angular velocity of the interventional surgical instrument motion commands generated by the master end for the execution end.

Said master end control box further includes: a controller; said controller communicates via said communication device with said linear motor, said displacement sensor, said DC motor/rotational electromagnetic damper, said rotary encoder, and said execution end, said controller controls said linear motor based on the resistance obtained by the execution end to provide axial feedback resistance for the axial movement of said operating lever, said controller controls said DC motor or rotational electromagnetic damper based on the resistive torque obtained by the execution end to provide rotational feedback resistive torque for the rotation of said operating lever, and said controller controls said execution end based on said axial displacement direction, said axial displacement distance, and said rotation angle.

As shown in FIGS. 2-13, the interventional surgical execution end 1000 according to an embodiment of the present disclosure includes a plurality of first friction wheels 1011 and a plurality of second friction wheels 1012.

The plurality of first friction wheels 1011 are arranged along a first horizontal direction (up-down direction as indicated by the arrow in the drawings, horizontal direction perpendicular to the up-down direction). The plurality of second friction wheels 1012 are arranged along the second horizontal direction. The plurality of first friction wheels 1011 are correspondingly and oppositely disposed relative to the plurality of second friction wheels 1012 in a second horizontal direction and are adapted to jointly grip an interventional surgical instrument. The plurality of first friction wheels 1011 and the plurality of second friction wheels 1012 are provided with helices 1013. The helix angles of the plurality of helices 1013 are all the same. The helical directions of the helices 1013 on adjacent two first friction wheels 1011 are opposite. The helical directions of the helices 1013 on adjacent two second friction wheels 1012 are opposite. The helical directions of the helices on opposing first friction wheel 1011 and second friction wheel 1012 are opposite.

In an exemplary embodiment of the present disclosure, the interventional surgical instrument may include guidewires, catheters, balloons, thrombectomy devices, etc.

The first horizontal direction and the second horizontal direction are not coincident; here, the first horizontal direction and the second horizontal direction are preferably parallel to each other.

The plurality of first friction wheels 1011 are adapted to rotate at the same speed and in the same direction. The plurality of second friction wheels 1012 are adapted to rotate at the same speed and in the same direction. And the rotation direction of the first friction wheels 1011 is opposite to that of the second friction wheels 1012.

Through the rotation of the first friction wheels 1011 and the second friction wheels 1012, the gripped interventional surgical instrument can be moved along its own axial direction, achieving delivery drive of the interventional surgical instrument.

The size of the helix angle of the helices 1013 can be adjusted according to actual needs while ensuring that the helix angles of the helices 1013 of all friction wheels are the same, to achieve different kinematic characteristics.

Taking both the first friction wheels 1011 and the second friction wheels 1012 as three, and the interventional surgical instrument as a guidewire as an example. Each opposing pair of first friction wheel 1011 and second friction wheel 1012 is a group. Multiple groups of friction wheels are distributed along the first horizontal direction into a first group, a second group, and a third group. Taking the first group of friction wheels as an example, the forward speed of the guidewire is denoted as u, the rotational angular velocity as ω, and the average radius of the guidewire as r. Then the velocity vector at the contact point between the guidewire and the first friction wheel 1011 is (u, ωᵣ). The velocity vector at the contact point between the guidewire and the second friction wheel 1012 is (u, −ωᵣ). The motion velocity of the guidewire contact point along the axial direction of the first friction wheel 1011 is ωᵣ. The motion velocity of the guidewire contact point along the axial direction of the second friction wheel 1012 is −ωᵣ. The two directions are opposite. As shown in FIG. 18a, taking the first friction wheel 1011 as an example, assuming its helix angle is α, the velocity of the guidewire contact point can be projected onto the velocity u₁ parallel to the helical direction of the helix 1013 and the velocity v₁ perpendicular to the helical direction. Their specific magnitudes are:

$${u}_{1} = u \times cos α + {ω}_{r} \times sin α ;$$

$${v}_{1} = - u \times sin α + {ω}_{r} \times cos α .$$

As shown in FIG. 18b, for the second friction wheel 1012 opposite to the first friction wheel 1011, its helical direction is opposite, and its helix angle is also α. The velocity of the guidewire contact point can also be decomposed via vector decomposition into a velocity u₂ parallel to the helical direction and a velocity v₂ perpendicular to the helical groove direction. The magnitudes of u₂ and v₂ are the same as those of u₁ and v₁ respectively. Therefore, for the same pair of friction wheels, the motion relationship between the friction wheels on both sides and the guidewire is the same, for avoiding the phenomenon of one side being tightly fitted while the other side experiences slippage.

As shown in FIG. 18c, the second group of friction wheels has the opposite helical direction to the first group, where:

$${u}_{3} = u \times cos α - {ω}_{r} \times sin α ;$$

$${v}_{3} = u \times sin α + {ω}_{r} \times cos α .$$

The magnitudes of u₄ and v₄ are the same as those of u₃, v₃. The helical directions of the helices 1013 of the first group of friction wheels and the third group of friction wheels are the same. The second group of friction wheels is opposite to both the first group and the third group. Therefore, regardless of the operating state of the friction wheels, it can be ensured that adjacent friction wheels experience resistance in opposite directions, preventing the guidewire from moving up and down.

According to the interventional surgical execution end 1000 of the embodiment of the present disclosure, by providing a plurality of first friction wheels 1011 and a plurality of second friction wheels 1012, with helices 1013 provided on both the plurality of first friction wheels 1011 and the plurality of second friction wheels 1012, the helix angles of the plurality of helices 1013 all being the same, the helical directions of the helices 1013 on adjacent two first friction wheels 1011 being opposite, the helical directions of the helices 1013 on adjacent two second friction wheels 1012 being opposite, and the helical directions of the helices on opposing first friction wheel 1011 and second friction wheel 1012 being opposite, relative slippage between the gripped interventional surgical instrument and the friction wheels can be prevented. Compared with the execution ends of interventional surgical instrument operating systems in the related art, the driving effect and accuracy of the interventional surgical execution end 1000 on the interventional surgical instrument can be improved. Moreover, for interventional surgical instrument operating systems with resistance feedback function, the resistance feedback can be made more accurate, allowing the doctor to more reliably judge the motion state of the interventional surgical instrument, improving the reliability of the surgical procedure.

Therefore, the interventional surgical execution end 1000 according to the embodiment of the present disclosure can prevent slippage of the interventional instrument and has advantages such as good driving effect, high accuracy, and strong reliability

Specific, embodiments of the interventional surgical execution end 1000 according to the present disclosure are described below with reference to the drawings.

In some specific embodiments of the present disclosure, as shown in FIGS. 2-13, the interventional surgical execution end 1000 according to an embodiment of the present disclosure includes a plurality of first friction wheels 1011 and a plurality of second friction wheels 1012.

In some embodiments, as shown in FIGS. 2-10, the interventional surgical execution end 1000 further includes a first lifting base 1021 and a second lifting base 1022. The plurality of first friction wheels 1011 are provided on the first lifting base 1021, and the first lifting base 1021 is liftable. The plurality of second friction wheels 1012 are provided on the second lifting base 1022, and the second lifting base 1022 is liftable. The rotation drive device is respectively connected in drive to the first lifting base 1021 and the second lifting base 1022 and is adapted to drive the first lifting base 1021 and the second lifting base 1022 to lift and lower in opposite directions. In other words, the rotation drive device drives the first lifting base 1021 to rise while driving the second lifting base 1022 to lower, and drives the first lifting base 1021 to lower while driving the second lifting base 1022 to rise. This allows the use of the opposite lifting and lowering of the first lifting base 1021 and the second lifting base 1022 to make the first friction wheels 1011 and the second friction wheels 1012 move up and down in opposite directions, thereby driving the interventional surgical instrument gripped by the first friction wheels 1011 and the second friction wheels 1012 to rotate circumferentially, achieving rotational drive of the interventional surgical instrument.

That is to say, the delivery drive of the interventional surgical instrument can be achieved through the rotation of the first friction wheels 1011 and the second friction wheels 1012, and the rotational drive of the interventional surgical instrument can be achieved through the opposite axial lifting and lowering of the first friction wheels 1011 and the second friction wheels 1012. This facilitates simultaneous delivery and rotational motion of the interventional surgical instrument, better conforms to the doctor's operation habits and intuition, facilitates reproducing the scenario of directly operating the interventional surgical instrument, allows doctors to apply existing experience, avoids mode switching in thinking, facilitates doctor operation, and moreover, facilitates more complex motion of the interventional surgical instrument, improving its ability to enter complex vasculature.

In an exemplary embodiment of the present disclosure, the interventional surgical execution end 1000 further includes a first upper limit switch, a first lower limit switch, a second upper limit switch, and a second lower limit switch. The first upper limit switch, first lower limit switch, second upper limit switch, and second lower limit switch are all electrically connected to the rotation drive device. When the first lifting base 1021 rises to the upper limit position, it triggers the first upper limit switch. When the first lifting base 1021 lowers to the lower limit position, it triggers the first lower limit switch. When the second lifting base 1022 rises to the upper limit position, it triggers the second upper limit switch. When the second lifting base 1022 lowers to the lower limit position, it triggers the second lower limit switch, to timely control the rotation drive device to stop driving.

Thrust ball bearings may be provided between the first friction wheels 1011 and the first lifting base 1021 and between the second friction wheels 1012 and the second lifting base 1022 to reduce friction.

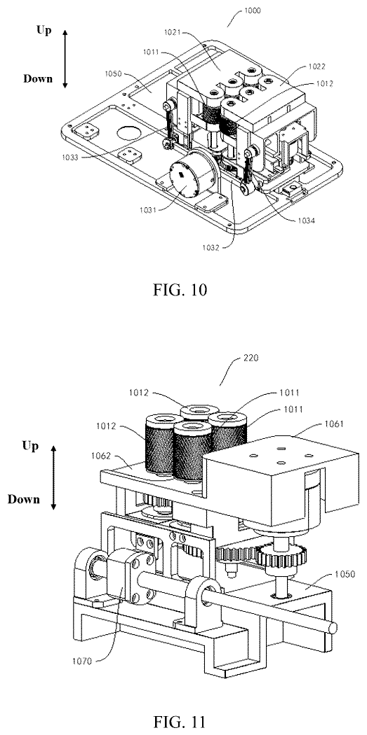

In an exemplary embodiment of the present disclosure, as shown in FIG. 10, the rotation drive device includes an up-down motion drive motor 1031, a crank 1032, a first connecting lever 1033, and a second connecting lever 1034. The center of the crank 1032 is connected to the motor shaft of the up-down motion drive motor 1031. Both ends of the first connecting lever 1033 are pivotally connected to one end of the crank 1032 and the first lifting base 1021, respectively. Both ends of the second connecting lever 1034 are pivotally connected to the other end of the crank 1032 and the second lifting base 1022, respectively. In an exemplary embodiment of the present disclosure, the up-down motion drive motor 1031 is a motor with a gearbox mechanism to reduce speed and increase torque. The form of the gearbox mechanism can be a planetary gear reducer, or cycloidal pinwheel reducer, etc. When the up-down motion drive motor 1031 rotates, one end of the crank 1032 rises and the other end lowers. The rising end drives the connected one of the first connecting lever 1033 and the second connecting lever 1034 to rise, and the lowering end drives the connected other one of the first connecting lever 1033 and the second connecting lever 1034 to lower, thereby further driving one of the first lifting base 1021 and the second lifting base 1022 to rise and driving the other one of the first lifting base 1021 and the second lifting base 1022 to lower, achieving opposite lifting and lowering of the first lifting base 1021 and the second lifting base 1022.

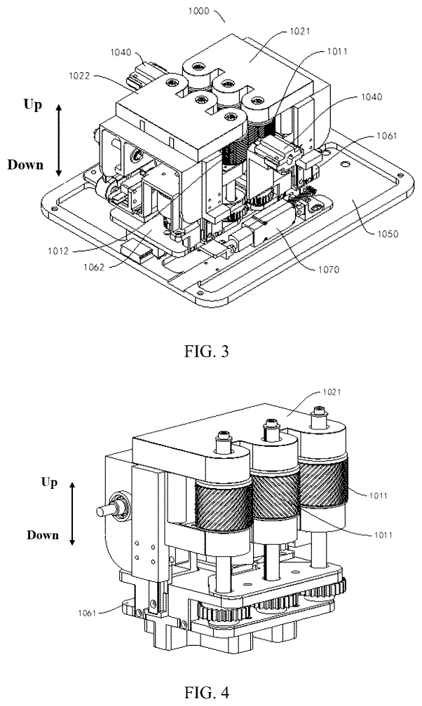

Advantageously, as shown in FIG. 3, the interventional surgical execution end 1000 further includes a radial limiting element 1040. The interventional surgical instrument is adapted to fit within the radial limiting element 1040 so as to be axially movable and circumferentially rotatable. The radial limiting element 1040 is adapted to limit radial movement of the interventional surgical instrument. In an exemplary embodiment of the present disclosure, the radial limiting element 1040 may include a groove body and a cover plate, both provided with grooves. The interventional surgical instrument is adapted to fit within the grooves. The cover plate is detachably mounted on the groove body to facilitate installation of the interventional surgical instrument. This can prevent the interventional surgical instrument from moving radially under the drive of the first friction wheels 1011 and the second friction wheels 1012, especially during rotational drive, ensuring that the interventional surgical instrument can only perform axial movement and circumferential rotation, i.e., delivery motion and rotational motion, further improving the reliability of driving the interventional surgical instrument.

In an exemplary embodiment of the present disclosure, the radial limiting element 1040 is detachable for easy replacement. The radial limiting element 1040 may be for single use, facilitating cleaning and maintenance of the interventional surgical instrument operating system 1.

In an exemplary embodiment of the present disclosure, as shown in FIGS. 2-12, the interventional surgical execution end 1000 further includes a base 1050, a fixed base 1061, a movable base 1062, and a gap adjustment drive device 1070. The fixed base 1061 is provided on the base 1050. The plurality of first friction wheels 1011 are provided on the fixed base 1061. The movable base 1062 is movably provided on the base 1050 along the second horizontal direction. The plurality of second friction wheels 1012 are provided on the movable base 1062. The gap adjustment drive device 1070 is provided on the base 1050 and is connected in drive to the movable base 1062. In an exemplary embodiment of the present disclosure, the gap adjustment drive device 1070 may be manually driven, such as a leadscrew-slider structure, or may be electrically controlled, such as a cylinder, push lever motor, rack and pinion structure, etc. The movable base 1062 may be movably provided on the base plate 1050 via a guide rail-slider structure. The height positions of the movable base 1062 and the fixed base 1061 can be adjusted according to the thickness of the guide rail slider to ensure that the first friction wheels 1011 and the second friction wheels 1012 are at the same height in the horizontal plane. This allows the gap adjustment drive device 1070 to drive the movable base 1062 to adjust the gap size between the fixed base 1061 and the movable base 1062, thereby adjusting the gap size between the first friction wheels 1011 and the second friction wheels 1012. For example, the gap between the first friction wheels 1011 and the second friction wheels 1012 can first be increased, then the interventional surgical instrument can be placed, and then the gap can be reduced to facilitate installation of the interventional surgical instrument. It can also adapt to interventional surgical instruments of different sizes by adjusting the gap between the first friction wheels 1011 and the second friction wheels 1012, improving the applicability of the interventional surgical execution end 1000.

In an exemplary embodiment of the present disclosure, the first lifting base 1021 is provided on the fixed base 1061. The second lifting base 1022 is provided on the movable base 1062. The plurality of first friction wheels 1011 are provided on the first lifting base 1021. The plurality of second friction wheels 1012 are provided on the second lifting base 1022.

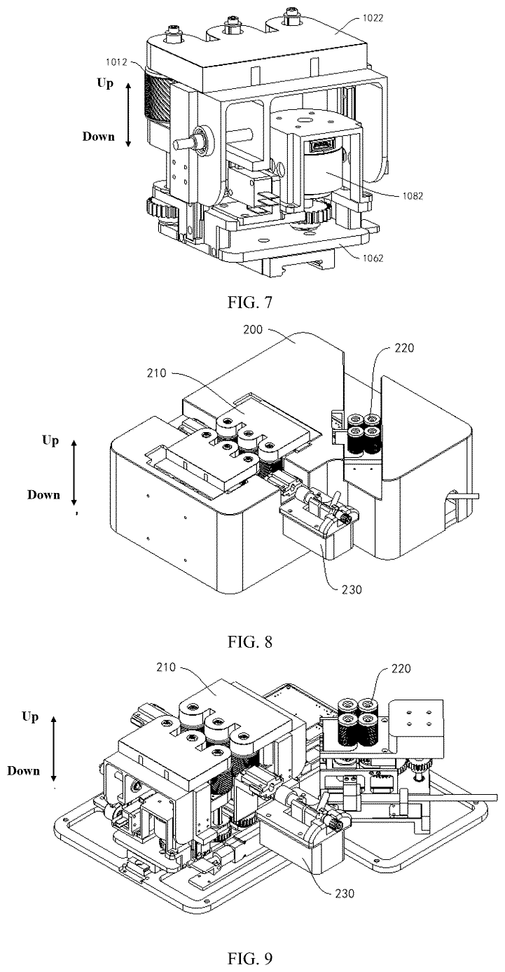

In some embodiments, as shown in FIGS. 5 and 7, the interventional surgical execution end 1000 further includes a first rotation drive device 1081 and a second rotation drive device 1082. The first rotation drive device 1081 is respectively connected in drive to the plurality of first friction wheels 1011. The first rotation drive device 1081 is adapted to drive the plurality of first friction wheels 1011 to rotate at the same speed and in the same direction. The second rotation drive device 1082 is respectively connected in drive to the plurality of second friction wheels 1012. The second rotation drive device 1082 is adapted to drive the plurality of second friction wheels 1012 to rotate at the same speed and in the same direction, and the rotation direction of the plurality of first friction wheels 1011 is opposite to that of the plurality of second friction wheels 1012. This allows the use of the first rotation drive device 1081 to drive the plurality of first friction wheels 1011, and the use of the second rotation drive device 1082 to drive the plurality of second friction wheels 1012, facilitating the same-speed and same-direction rotation of the first friction wheels 1011, facilitating the same-speed and same-direction rotation of the second friction wheels 1012, and facilitating the opposite rotation of the first friction wheels 1011 and the second friction wheels 1012.

In an exemplary embodiment of the present disclosure, the first rotation drive device 1081 is provided on the fixed base 1061. The second rotation drive device 1082 is provided on the movable base 1062.

The first friction wheels 1011 are detachably provided, and the second friction wheels 1012 are detachably provided, for easy replacement. The first friction wheels 1011 and the second friction wheels 1012 may both be for single use, to facilitate cleaning and maintenance of the interventional surgical instrument operating system 1.

As shown in FIG. 13, each friction wheel is detachably mounted on a friction wheel base 1016 to facilitate replacement of the friction wheels. A cross slot may be used between the friction wheel and the friction wheel base 1016 to restrict relative rotation.

The friction wheel base 1016 is axially movably provided on a flat key shaft 1014 and rotates together with the flat key shaft 1014, to facilitate driving the friction wheel while allowing it to move up and down. A driven gear 1015 may be connected to the flat key shaft 1014. Adjacent two driven gears 1015 may both mesh with a transmission gear. The transmission gear meshes with a drive gear connected to the rotation drive device. The modules and number of teeth of the plurality of driven gears 1015 and the transmission gear are all equal, to facilitate multiple friction wheels driven by one rotation drive device to rotate at the same speed and direction.

A baffle plate may be provided above the driven gear 1015 to prevent liquid from the surgery from entering the transmission part and affecting the transmission effect.

In some embodiments, as shown in FIGS. 2-10, both the first friction wheels 1011 and the second friction wheels 1012 may be three, to simulate three-finger operation of the human hand.

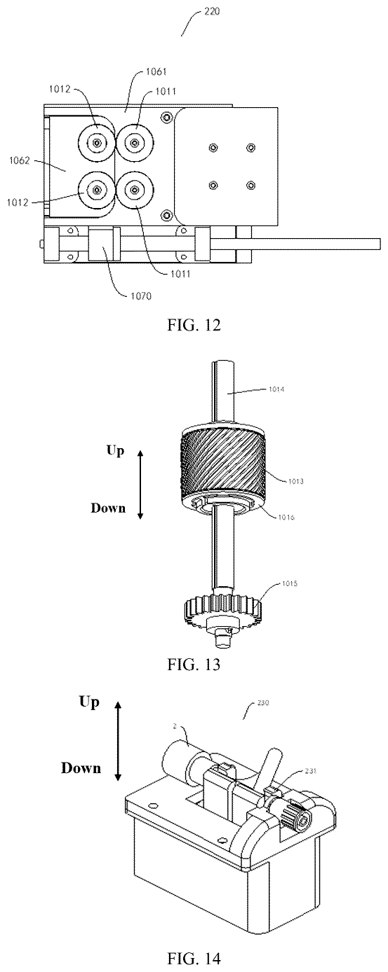

In other embodiments, as shown in FIG. 11, both the first friction wheels 1011 and the second friction wheels 1012 may be two, and the rotation drive device may also be one and connected in drive to the plurality of first friction wheels 1011 and the plurality of second friction wheels 1012.

The execution end according to an embodiment of the present disclosure is described in detail below. The execution end according to an embodiment of the present disclosure includes a catheter operation module 110 and/or a guidewire operation module 210.

The catheter operation module 110 includes the interventional surgical execution end 1000 according to the above-described embodiment of the present disclosure.

The guidewire operation module 210 includes the interventional surgical execution end 1000 according to the above-described embodiment of the present disclosure.

The execution end according to the embodiment of the present disclosure, by utilizing the interventional surgical execution end 1000 according to the above-described embodiment of the present disclosure, can prevent slippage of the interventional instrument and has advantages such as good driving effect, high accuracy, and strong reliability.

In an exemplary embodiment of the present disclosure, as shown in FIGS. 1, 15, and 16, the execution end further includes a roller cart 400 and a passive damping mechanical arm 300. The roller cart 400 includes a support base 410, a support lever 420, and rollers 430. The support lever 420 is provided on the support base 410. The rollers 430 are provided on the lower surface of the support base 410. In an exemplary embodiment of the present disclosure, the rollers 430 are plural and have damping structures and locking devices to facilitate stopping the roller cart 400 on the ground and prevent the roller cart 400 from moving arbitrarily. The passive damping mechanical arm 300 is provided on the support lever 420. The passive damping mechanical arm 300 includes at least two arm segments 310. The catheter operation module 110 and the guidewire operation module 210 are respectively provided on the two arm segments 310. This allows the entire execution end to be moved by moving the roller cart 400, and by operating the passive damping mechanical arm 300, the specific positions of the catheter operation module 110 and the guidewire operation module 210 can be adjusted to facilitate finding a suitable surgical position.

In an exemplary embodiment of the present disclosure, the passive damping mechanical arm 300 may include a connection base 320, a support tube 330, a connection tube 340, and two arm segments 310. Each arm segment 310 may include an adapter base 311, a first bent tube 312, a second bent tube 313, and a third bent tube 314. The connection base 320 is connected to the upper end of the support lever 420 of the roller cart 400. The lower end of the support tube 330 is rotatably connected to the connection base 320 via a damping joint. The middle part of the connection tube 340 is rotatably connected to the upper end of the support tube 330 via a damping joint. The two ends of the connection tube 340 are rotatably connected to the third bent tubes 314 of the two arm segments 310 respectively via damping joints. The second bent tube 313 is rotatably connected to the third bent tube 314 via a damping joint. The first bent tube 312 is rotatably connected to the second bent tube 313 via a damping joint. The adapter base 311 is rotatably connected to the first bent tube 312 via a damping joint. The two adapter bases 311 are respectively connected to the catheter operation module 110 and the guidewire operation module 210. This gives the passive damping mechanical arm 300 multiple degrees of freedom in various directions, and it can hover via the damping joints, facilitating adjustment of the posture of the catheter operation module 110 and the guidewire operation module 210.

The execution end according to an embodiment of the present disclosure includes a catheter operation module 110, a guidewire operation module 210, and a balloon catheter operation module 220. The catheter operation module 110 is provided on a catheter operation box 100 and is adapted to operate a catheter. The guidewire operation module 210 and the balloon catheter operation module 220 are provided on a guidewire/balloon operation box 200 and are respectively adapted to operate a guidewire and a balloon catheter. The catheter operation box 100 is connected to the adapter base 311 of one of the arm segments 310. The guidewire/balloon operation box 200 is connected to the adapter base 311 of the other arm segment 310.

The guidewire/balloon operation box 200 is further provided with a Y-valve positioning device 230, and a Y-valve 2 is adapted to be mounted on the Y-valve positioning device 230. The Y-valve positioning device 230 is provided with an elastic claw 231. The elastic claw 231 may be provided with elasticity by a torsion spring. One side of the elastic claw 231 is provided with an avoidance groove to avoid the branch tube of the Y-valve 2.

On the guidewire/balloon operation box 200, the delivery direction of the balloon catheter operation module 220 is arranged at a certain angle to the delivery direction of the guidewire operation module 210 to facilitate adaptation to the Y-valve 2.

The catheter operation module 110, guidewire operation module 210, and balloon catheter operation module 220 may respectively adopt the same or different implementations of the above-described interventional surgical execution end 1000.

In some embodiments, as shown in FIGS. 2-7, the interventional surgical execution end 1000 of the catheter operation module 110 needs to perform rotation and delivery operations, so it needs to include a plurality of first friction wheels 1011, second friction wheels 1012, a first lifting base 1021, a second lifting base 1022, a rotation drive device, a base 1050, a fixed base 1061, a movable base 1062, a gap adjustment drive device 1070, a first rotation drive device 1081, and a second rotation drive device 1082.

As shown in FIGS. 8-10, the interventional surgical execution end 1000 of the guidewire operation module 210 also needs to perform rotation and delivery operations, and may similarly include a plurality of first friction wheels 1011, second friction wheels 1012, a first lifting base 1021, a second lifting base 1022, a rotation drive device, a base 1050, a fixed base 1061, a movable base 1062, a gap adjustment drive device 1070, a first rotation drive device 1081, and a second rotation drive device 1082.

As shown in FIGS. 11 and 12, the interventional surgical execution end 1000 of the balloon catheter operation module 220 may only perform delivery operations, and may include a plurality of first friction wheels 1011, second friction wheels 1012, a base 1050, a fixed base 1061, a movable base 1062, and a gap adjustment drive device 1070.

The balloon catheter operation module 220 may include one rotation drive device and be adapted to be connected in drive to the plurality of first friction wheels 1011 and the plurality of second friction wheels 1012 via a gear set. Its movable base 1062 may have a separated position and an engaged position. When the movable base 1062 is in the separated position, the gear connected to the first friction wheels 1011 is disengaged from the gear connected to the second friction wheels 1012. When the movable base 1062 is in the engaged position, the gear connected to the first friction wheels 1011 meshes with the gear connected to the second friction wheels 1012 to achieve transmission. This allows the movable base 1062 to be moved to the separated position when installing the balloon catheter, and then moved back to the engaged position after installation is complete.

The gap adjustment drive device 1070 of the balloon catheter operation module 220 may be manually adjusted, such as a rotary knob, spherical joystick, push-pull handle, etc.

The interventional surgical instrument operating system 1 according to an embodiment of the present disclosure is described in detail below. The interventional surgical instrument operating system 1 according to an embodiment of the present disclosure includes a master end 20, a communication device 30, and an execution end.

The execution end is the execution end according to the above-described embodiment of the present disclosure, and the execution end communicates with the master end 20 via the communication device 30.

In an exemplary embodiment of the present disclosure, the communication device 30 may be a wired communication device or a wireless communication device.

The interventional surgical instrument operating system 1 according to the embodiment of the present disclosure, by utilizing the execution end according to the above-described embodiment of the present disclosure, can achieve composite linear and rotational motion of the interventional surgical instrument, i.e., the ideal state of push-twist motion, prevent slippage of the interventional instrument, and has advantages such as simulating manual operation by doctors, high accuracy, and strong reliability.

Furthermore, doctors can operate the master end 20 outside the operating room or even remotely, and remotely control the execution end via the communication device 30 to perform surgery on the patient. This method allows doctors to be free from X-ray radiation, protecting their own health. At the same time, due to its remote control characteristic, doctors can also perform remote surgeries across regions, which helps improve treatment levels in remote and medically underdeveloped areas, saving doctors' travel time.

Doctors personally operate the master end 20, which reads and measures the doctor's operations and sends this data to the execution end via the communication device 30. The two execution ends receive motion control information and then perform corresponding push, pull, and rotation operations of the interventional surgical instrument according to the control information flow, thereby completing the surgery.

In an exemplary embodiment of the present disclosure, the execution end is adapted to detect the delivery resistance and rotational resistance experienced by the interventional surgical instrument. In an exemplary embodiment of the present disclosure, the rotation drive device and the rotation drive device may have force feedback functions, for example, they may be force feedback motors.

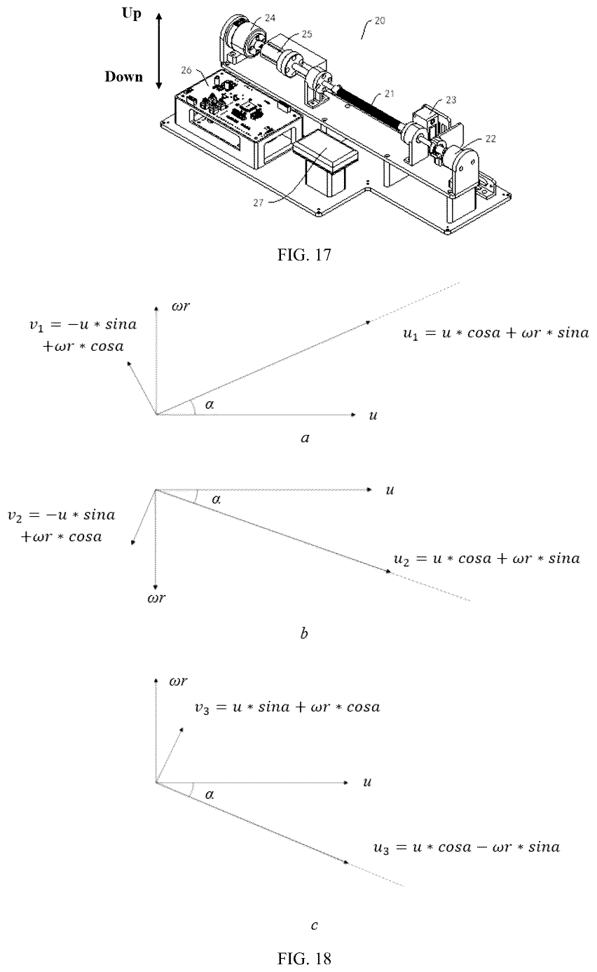

Advantageously, as shown in FIG. 17, the master end 20 includes an operating lever 21, a linear motor 22, a displacement detection device 23, a drive motor 24, an angle detection device, and a controller 26.

The operating lever 21 is axially movable and rotatable about its central axis. The linear motor 22 is connected to the operating lever 21. The displacement detection device 23 is adapted to detect the axial displacement direction and axial displacement distance of the operating lever 21. The drive motor 24 is connected to the operating lever 21 via a ball spline structure 25. The angle detection device is adapted to detect the rotation angle of the operating lever 21. The controller 26 is respectively electrically connected to the linear motor 22, displacement detection device 23, drive motor 24, and the angle detection device, and is adapted to communicate with the execution end via the communication device 30.

The controller 26 is adapted to control the linear motor 22 based on the delivery resistance to provide axial feedback resistance for the axial movement of the operating lever 21. The controller 26 is adapted to control the drive motor 24 based on the rotational resistance to provide rotational feedback resistance for the rotation of the operating lever 21. The controller 26 is adapted to control the execution end based on the axial displacement direction, axial displacement distance, and rotation angle.

In an exemplary embodiment of the present disclosure, the linear motor 22 may be a linear motor. The displacement detection device 23 may be a laser displacement sensor, ultrasonic displacement sensor, radar, grating scale, etc. Here, a laser displacement sensor is taken as an example. The angle detection device may be provided on the drive motor 24. The master end 20 further includes a hand support pad 27 adapted to support the doctor's hand.

The doctor performs axial movement and rotation operations on the operating lever 21, which can be performed simultaneously. The displacement detection device 23 and the angle detection device can read the rotation angle and positive/negative displacement magnitude of the axial movement of the operating lever 21. The signals are sent to the execution end via the controller 26 and the communication device 30 to drive the interventional surgical instrument to perform corresponding operations.

The delivery resistance and rotational resistance detected by the execution end are sent back to the controller 26 via the communication device 30. The controller 26 drives the linear motor 22 and the drive motor 24 to provide resistance in the corresponding directions respectively, to simulate the resistance experienced by the interventional surgical instrument on the operating lever 21.

The ball spline structure 25 can decouple the linear and rotational motions of the operating lever 21. A ball spline nut is fitted over a ball spline shaft. The ball spline shaft is connected to the operating lever 21. The ball spline nut is connected to the rotor part of the drive motor 24. This ensures that the linear motion of the operating lever 21 does not affect the drive motor 24; only rotational motion is transmitted to the drive motor 24.

The controller 26 may have a display and control interface, which can display the operating status and parameters of the interventional surgical instrument operating system 1. The display and control interface may be provided with a function switching device and a proportional adjustment device. The function switching device is adapted to switch the operation module controlled by the master end 20 among the catheter operation module 110, guidewire operation module 210, and balloon catheter operation module 220. The proportional adjustment device can adjust the ratio between the motion amplitude of the operating lever 21 and the motion amplitude of the interventional surgical instrument driven by the execution end.

The operation process of the interventional surgical instrument operating system 1 according to an embodiment of the present disclosure is described below with reference to FIGS. 1-17. The execution end is placed in the operating room and communicates with the master end 20 via the communication device 30. The doctor installs the catheter on the catheter operation box 100, and installs the guidewire, angiographic catheter, and balloon catheter onto the corresponding positions of the guidewire/balloon operation box 200. The doctor operates the operating lever 21 of the master end 20. The hand operations are recorded and read by the master end 20. The operation data is decomposed into forward/backward delivery displacement and twisting motion rotation angle and transmitted via the communication device 30 to the catheter operation module 110, guidewire operation module 210, and balloon catheter operation module 220 within the execution end. The controller 26 has a function switching device, which can determine the interventional surgical execution end 1000 controlled by the master end 20. The doctor's forward/backward delivery operation on the operating lever 21 can control the displacement speed magnitude and direction of the operated interventional surgical instrument at the execution end. The doctor's twisting operation on the operating lever 21 can control the rotation angle magnitude and direction of the operated interventional surgical instrument at the execution end. These two operations can be performed synchronously and in real-time, achieving remote operation by the doctor outside the operating room.

While the doctor controls the motion of the interventional surgical instrument at the execution end, the resistance and resistive torque experienced by the interventional surgical instrument at the execution end are also recorded by the drive devices of the execution end and sent to the master end 20 via the communication device 30. This resistance and resistive torque data is used to implement force feedback via the linear motor 22 and drive motor 24 of the master end 20. The resistance for the feed motion is implemented by the linear motor 22, and the resistive torque for the rotational motion is implemented by the drive motor 24. Therefore, during the doctor's actual operation, while holding and operating the operating lever 21 to control the motion of the operated interventional surgical instrument at the execution end, the force state of the operated interventional surgical instrument at the execution end is also fed back to the doctor via the master end 20. Furthermore, the doctor's forward/backward delivery operation and twisting operation can be completed simultaneously with one operating lever 21, without needing two separate joysticks, thus achieving an intuitive operation experience and lowering the threshold for doctors to start operating.

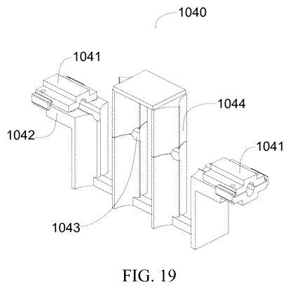

When a pair of opposing friction wheels move relatively up and down to rotate the interventional surgical instrument, they may also cause the interventional surgical instrument to move up and down. To avoid this, FIG. 19 shows an interventional surgical instrument placement guiding device 1040 for preventing radial up-down motion of the interventional surgical instrument according to an embodiment of the present disclosure. In the drawings, 1042 and 1043 are holes for the interventional surgical instrument to pass through, formed by grooves in the upper and lower cover plate structures.

The guiding device is divided into a plurality of modules, each module including an upper cover and a base 1042. The bases of all modules are connected to each other to form an integral whole. The middle of the base and the upper cover has a groove, and two opposite grooves form a hole 1043. The interventional surgical instrument passes through from the middle. The width of the groove is larger than the diameter of the interventional surgical instrument placed thereon. The depth of the groove is larger than the radius of the interventional surgical instrument placed thereon. 1041 is the upper cover plate, inserted from the outside to connect the basic structure 1042 of multiple modules. 1044 is the cover plate of the intermediate structure, directly inserted vertically into the basic structure 1042.

An embodiment shown in FIG. 19 includes three said modules. The first module is installed between pairs of friction wheels arranged side by side along the first horizontal direction. The base and the upper cover form a three-dimensional structure. The upper cover connects a plurality of upright structures located along the second horizontal direction between adjacent friction wheels arranged along the first horizontal direction. The second and third module structures are respectively installed outside the sides of the plurality of friction wheels arranged side by side along the first horizontal direction and the second horizontal direction.

For complex vascular networks, it is often necessary to reduce the forward speed and rotational angular velocity of the interventional surgical instrument. By proportionally reducing the linear motion and rotation angle amplitudes generated by the master end, operational refinement can be achieved. To this end, a scaling factor device for proportionally amplifying or reducing the linear motion amplitude, linear motion speed, rotation angle, and rotational angular velocity of the interventional surgical instrument motion commands generated by the master end for the execution end is installed on the master end control box. One implementation method is to use two knobs to define the linear motion scaling factor and rotational motion scaling factor respectively. One definition of the scaling factor is from 1× to 0.1× divided into ten proportions.

Other components and operations of the interventional surgical instrument operating system 1 according to the embodiment of the present disclosure are known to those of ordinary skill in the art and will not be described in detail here.

In the description of this specification, descriptions referring to terms such as “one embodiment,” “some embodiments,” “illustrative embodiment,” “example,” “specific example,” or “some examples” mean that specific features, structures, materials, or characteristics described in connection with the embodiment or example are included in at least one embodiment or example of the present disclosure. In this specification, schematic representations of the above terms do not necessarily refer to the same embodiment or example. Furthermore, the described specific features, structures, materials, or characteristics may be combined in any suitable manner in one or more embodiments or examples.

Although embodiments of the present disclosure have been shown and described, those of ordinary skill in the art can understand that various changes, modifications, substitutions, and variations can be made to these embodiments without departing from the principles and spirit of the present disclosure. The scope of the present disclosure is defined by the claims and their equivalents.

What is claimed is:

1. An execution end for an interventional surgical instrument operating system, the execution end comprising:
a plurality of friction wheels configured to grip and convey an interventional surgical instrument,
wherein an outer surface of each of the plurality of friction wheels has grooves forming a plurality of helices with an acute helix angle; and
wherein the grooves are step-shaped, constituting a layer of uneven helical grooves on the outer surface of the friction wheel.

2. The execution end for an interventional surgical instrument operating system according to claim 1, wherein the plurality of friction wheels comprise:
a plurality of first friction wheels arranged along a first horizontal direction;
a plurality of second friction wheels arranged along the first horizontal direction, each of the plurality of first friction wheels opposing a corresponding second friction wheel of the plurality of second friction wheels along a second horizontal direction,
wherein helical groove directions of the plurality of first friction wheels are opposite helical groove directions of the plurality of second friction wheels.

3. The execution end for an interventional surgical instrument operating system according to claim 1, further comprising a rotating spline shaft, the plurality of friction wheels being mounted on said rotating spline shaft,
wherein an interior of each of the plurality of friction wheels is gear-shaped, allowing the friction wheel to move up and down along the rotating spline shaft while performing a rotational motion.

4. The execution end for an interventional surgical instrument operating system according to claim 2, further comprising:
a fixed base, a movable base, lifting bases, and a bottom plate of an actuator housing, wherein:
the fixed base is secured to the bottom plate;
the movable base is mounted on a slide rail;
a first lifting base of the lifting base carries the plurality of first friction wheels, and a second lifting base of the lifting bases carries the plurality of second friction wheels, the first lifting base and the second lifting base moving vertically in opposite directions to generate a composite rotational-linear motion.

5. The execution end for an interventional surgical instrument operating system according to claim 2, further comprising:
a first rotational driving assembly with a motor and gear system, the first rotational driving assembly being configured to drive the plurality of first friction wheels;
a second rotational driving assembly with a motor and gear system, the second rotational driving assembly being configured to drive the plurality of second friction wheels in an opposite rotational direction.

6. The execution end for an interventional surgical instrument operating system according to claim 4, further comprising a friction wheel up-down motion drive device configured to convert a rotational motion of a drive motor into a linear up-down motion for driving the lifting bases via a pair of crank-link mechanisms, the friction wheel up-down motion drive device comprising:
a crank, a center of said crank being connected to a motor shaft of said drive motor;
a first connecting lever, both ends of said first connecting lever being pivotally connected to one end of said crank and said first lifting base respectively, driving said first lifting base to move up and down; and
a second connecting lever, both ends of said second connecting lever being pivotally connected to the other end of said crank and said second lifting base respectively, driving said second lifting base to move up and down.

7. The execution end for an interventional surgical instrument operating system according to claim 4, further comprising a gap adjustment drive device, said gap adjustment drive device comprising:
a linear motion actuator arranged parallel to said slide rail, a fixed part of the linear motion actuator being fixed on an inner surface of said bottom plate of the actuator housing, and an extendable part of the linear motion actuator being connected to a bottom of the movable base; and
a displacement sensor configured to record a distance between said movable base and said fixed base.

8. The execution end for an interventional surgical instrument operating system according to claim 4, further comprising:
a limit switch system configured to restrict an up-down motion of the plurality of friction wheels,
wherein when the plurality of friction wheels move up or down to reach a maximum stroke of the lifting bases on which the plurality of friction wheels are mounted, a limit switch is triggered to achieve automatic reset by an automatic reset execution end.

9. The execution end for an interventional surgical instrument operating system according to claim 7, wherein said gap adjustment drive device has an automatic reset function,
wherein when a limit switch is triggered, said gap adjustment drive device drives the movable base and the fixed base to be separated from each other, the displacement sensor records a separation distance, a friction wheel up-down motion drive device moves said first lifting base and said second lifting base to an initial motion position, and then the gap adjustment drive device moves the movable base back to a position before separation in a reverse direction according to the separation distance recorded by the displacement sensor, to grip the interventional surgical instrument again.

10. The execution end for an interventional surgical instrument operating system according to claim 1, further comprising a guiding device with multiple modules forming grooves and through-holes for stabilizing the interventional instrument, each module being installed between pairs of friction wheels of the plurality of friction wheels arranged side by side along a horizontal direction.

11. A method for measuring resistance and resistive torque experienced by an interventional surgical instrument, comprising:
detecting resistance and rotational resistive torque experienced by the interventional surgical instrument delivered by an execution end; and
measuring resistance comprising steps of:
measuring an input power of an up-down motion drive motor and a rotation drive motor before an interventional surgical instrument enters a patient's body as an initial input power value;
measuring the input power of the up-down motion drive motor and the rotation drive motor after the interventional surgical instrument enters the patient's body;
subtracting the initial input power value from the measured input power, and taking the obtained difference as a power consumed by the resistance experienced by the interventional surgical instrument; and
through a conversion formula, obtaining the resistance and the resistive torque experienced by the interventional surgical instrument after entering the patient's body, wherein the conversion formula is obtained through experiments.

12. An execution end, comprising:
a first interventional surgical instrument operation module, said first interventional surgical instrument operation module comprising the execution end for an interventional surgical instrument operating system according to claim 1; and/or
a second interventional surgical instrument operation module, said second interventional surgical instrument operation module comprising the execution end for an interventional surgical instrument operating system according to claim 1; and/or
a third interventional surgical instrument operation module, installed on the same base plate as the second interventional surgical instrument operation module, the third interventional surgical instrument operation module being configured to push interventional surgical instruments that do not require rotational twisting, the third interventional surgical instrument operation module comprising a plurality of third friction wheels arranged along a third horizontal direction and a plurality of fourth friction wheels arranged along a fourth horizontal direction, rotation directions of the plurality of third friction wheels being opposite to rotation directions of the plurality of fourth friction wheels, and the plurality of third friction wheels and the plurality of fourth friction wheels being driven by the same drive motor via a gear set.

13. The execution end according to claim 12, further comprising:
a roller trolley with support structures and an articulated lockable mechanical arm on which actuator modules are mounted.

14. An interventional surgical instrument operating system comprising:
a master end;
a communication device; and
the execution end according to claim 12.

15. The interventional surgical instrument operating system according to claim 14, wherein the master end comprises:
a circular operating lever;
a linear-motion module with a displacement sensor; and
a rotational-motion module with a rotary encoder.

16. The interventional surgical instrument operating system according to claim 15, wherein:
said circular operating lever is configured to simulate a torque device for gripping and operating the interventional surgical instrument, a surface of the circular operating lever being knurled or having groove patterns, a first end of the circular operating lever being connected to a moving part of a linear motor, a second end of the circular operating lever being connected to a rotating part of a DC motor or a rotational electromagnetic damper via a spline shaft, said circular operating lever being capable of moving back and forth along the spline shaft;
a distance by which said circular operating lever moves towards the linear motor defines a speed at which the execution end manipulates the interventional surgical instrument to move forward, a greater distance corresponding to a higher speed.

17. The interventional surgical instrument operating system according to claim 15, wherein the linear-motion module comprises a displacement sensor and a linear motor providing axial force feedback.

18. The interventional surgical instrument operating system according to claim 15, wherein the rotational-motion module comprises a rotary encoder and a DC motor or a rotational electromagnetic damper providing torque feedback.

19. The interventional surgical instrument operating system according to claim 15, wherein the master end further comprises a control box with scaling modules for proportionally amplifying or reducing a linear motion amplitude, a linear motion speed, a rotation angle, and a rotational angular velocity of interventional surgical instrument motion commands generated by the master end for the execution end.

20. The interventional surgical instrument operating system according to claim 19, wherein the control box further comprises a controller configured to communicate with motors and sensors, generate force and torque feedback and control the execution end according to an axial displacement and a rotational angle.
